(19) United States
(12) Patent Application Publication
(10) Pub. No.: US 2024/0122600 A1
(43) Pub. Date: Apr. 18, 2024

(54) EMBOLIZATION DEVICES AND RELATED METHODS OF MANUFACTURE AND USE

(71) Applicant: KA Medical, LLC, Roseville, MN (US)

(72) Inventors: John Oslund, Roseville, MN (US); Kevin Dunne, Roseville, MN (US)

(21) Appl. No.: 18/484,972

(22) Filed: Oct. 11, 2023

Related U.S. Application Data
(60) Provisional application No. 63/379,396, filed on Oct. 13, 2022.

(51) Int. Cl. A61B 17/12

(52) U.S. Cl. CPC A61B 17/12113; A61B 17/12172; A61B 2017/00526

(57) ABSTRACT

Devices used to restrict flow within a blood vessel are disclosed. Devices within the scope of this disclosure include a braided lattice of nitinol wires that form self-expanding enclosures of an embolic structure. The self-expanding enclosures may be sized and dimensioned such that when deployed within a blood vessel, at least one of the plurality of self-expanding enclosures takes on an elongated generally cylindrical shape having opposing generally conical, frustoconical, or semispherical end regions. Methods of manufacturing and using the embolic structures are disclosed.

### RELATED APPLICATIONS

This application claims priority to U.S. Provisional Application No. 63/379,396, filed on Oct. 13, 2022 and titled, “Embolization Devices, and Related Methods of Manufacture and Use,” which is hereby incorporated by reference in its entirety.

### TECHNICAL FIELD

The present disclosure generally relates to intravascular devices for treating certain medical conditions, including use of low-profile intravascular occlusion devices for treating vascular defects and/or to prevent blood flow within a vessel.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1A is a side view of an embodiment of an embolization device including an elongated enclosure deployed in a blood vessel.

FIG. 1B is a side view of an embodiment of an embolization device having an elongated enclosure deployed in a blood vessel.

FIG. 1C is a side view of the embodiment of the embolization device of FIG. 1A deployed in a curved blood vessel.

FIG. 2A is a cross-sectional view of an embodiment of a mold for forming the embodiment of the embolization device of FIG. 1A, according to an embodiment.

FIG. 2B is a side view of an embodiment of a braided lattice.

FIG. 2C is a side view of the embodiment of the embolization device of FIG. 1A in an expanded state.

FIGS. 3A and 3B are cross-sectional views of an embodiment of an embolization device including a cup-shaped member.

FIG. 3C is a side view of an embodiment of the cup-shaped member of the embodiment of the embolization device of FIGS. 3A and 3B.

FIGS. 3D and 3E are views of the embodiment of the embolization device of FIGS. 3A and 3B.

FIGS. 3F-3K are side views of embodiments of embolization devices including a cup-shaped member.

FIGS. 4A-4B are side views of an embodiment of an embolization device having two anchor members and an occlusion member deployed in different sized blood vessels.

FIG. 4C is a side view of an embodiment of an embolization device having an anchor member and three occlusion members deployed in a blood vessel.

FIG. 4D is a side view of the embodiment of the embolization device of FIG. 4C in an expanded state.

FIGS. 5A and 5B are cross-sectional views of molds for forming an embolization device having two anchor members and one occlusion member.

FIG. 6 is a perspective view an embodiment of an embolization device having two anchor members and one occlusion member in an expanded state.

FIGS. 7A-7C are views of an embodiment of an embolization device having multiple enclosures configured to at least partially nest within one another.

FIG. 7D is a side view of the embodiment of the embolization device of FIGS. 7A-7C deployed in a blood vessel.

### DETAILED DESCRIPTION

Intravascular devices are used in various medical procedures. For example, embolization devices may be used to treat arterial-venous malformations, aneurysms, and other vascular defects, or to prevent blood flow to tumors or other portions of the body.

In some instances, an embolization device includes an embolic structure comprising a plurality of enclosures or baskets. Enclosures within the scope of this disclosure include baskets of a braided lattice or matrix, including embodiments formed of nitinol wires. While reference is made herein to a braided lattice, in some embodiments, the present disclosure may be applied to baskets that include a woven lattice or matrix. The plurality of enclosures may be coupled together and may be releasably coupled to a placement wire. The enclosures can be crimped or constrained to a small diameter and disposed within a delivery catheter for deployment into a body lumen such as a blood vessel. In some embodiments, in a fully expanded configuration, one or more enclosures of an embolization device may have a disk shape or a partial disk-like shape with opposing sides disposed in a generally parallel arrangement. In a partially expanded state, one or more enclosures of an embolization device may be elongate, spherical, ovoid, cylindrical, or other shapes, as shall be described in greater detail below. The enclosures of some embodiments may be configured to restrict blood flow through the blood vessel when deployed within a blood vessel. When deployed the enclosure may be fully or partially expanded, including instances where the degree of expansion is controlled by interaction between the vessel wall and the enclosure.

Embolization devices within the scope of this disclosure can be manufactured by braiding or weaving filaments to create a lattice or basket defining the enclosure. Filaments within the scope of this disclosure include metals and polymers, including superelastic or shape memory materials. For example, nitinol wires may be used to form the embolic structure of the enclosures. In some embodiments, a continuous braid of filaments may be used to form a plurality of enclosures with necked down middle portions disposed between the enclosures. The embolic structure may be crimped to a small diameter to fit within a delivery catheter. In many embodiments, a continuous braid of filaments may be heat set while disposed within a mold to form a plurality of enclosures of an embolization device.

An embolization device may be used in procedures to occlude vascular structures such as blood vessels. The embolization device can be deployed into a blood vessel by positioning a guide catheter at a desired deployment location for the embolization device, inserting the delivery catheter into the guide catheter, deploying the embolic structure into the blood vessel, and releasing the embolic structure from a placement wire. Once deployed, in some embodiments, one or more of the enclosures of the embolic structure can self-expand until it contacts the vessel wall. When expanded, the braided lattice of the embolic structure may restrict blood flow through the blood vessel. In certain instances, the restricted blood flow through the blood vessel results in formation of a thrombus or clot within the blood vessel.

Embodiments may be understood by reference to the drawings. It will be readily understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings or figures, these are not necessarily drawn to scale unless specifically indicated.

Various features are sometimes grouped together in a single embodiment, figure, or description thereof for the purpose of streamlining the disclosure. Many of these features may be used alone and/or in combination with one another.

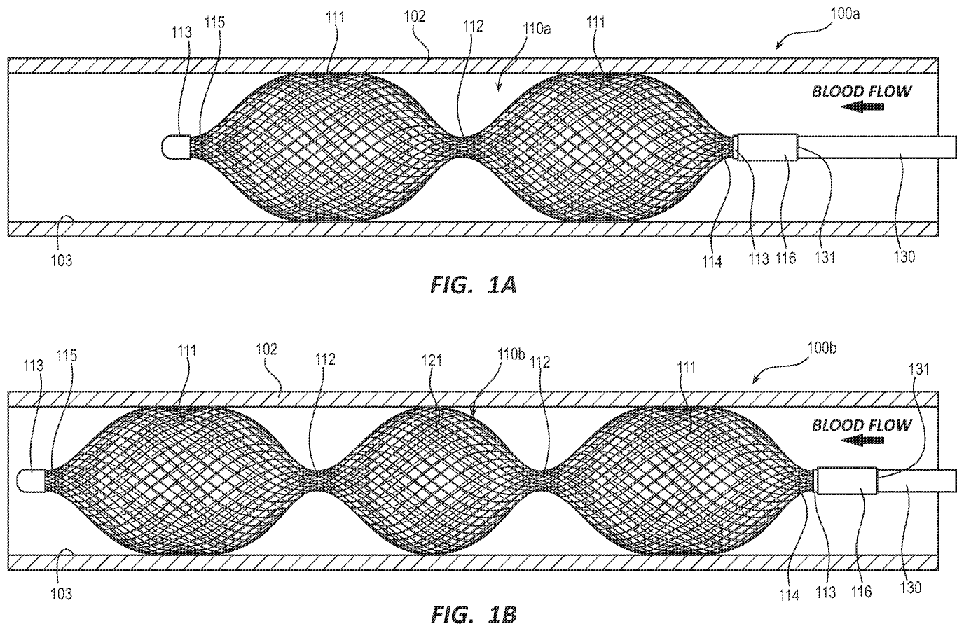

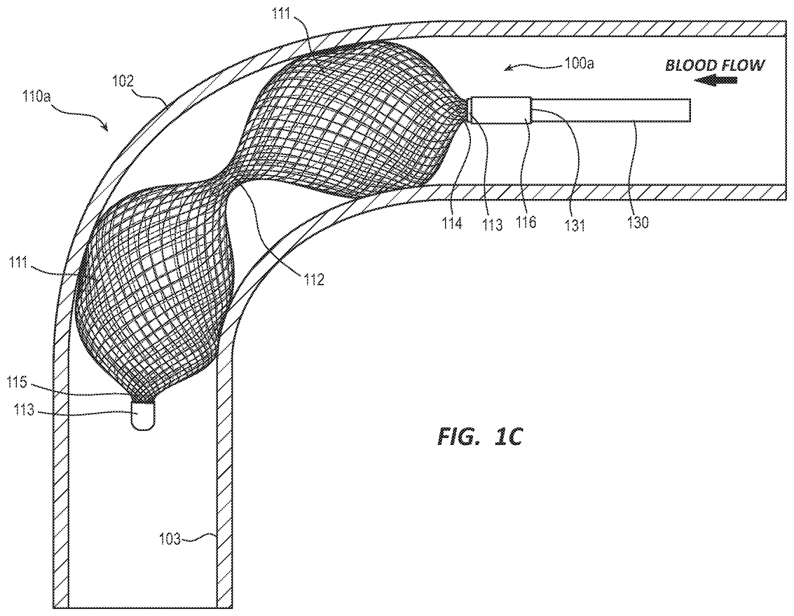

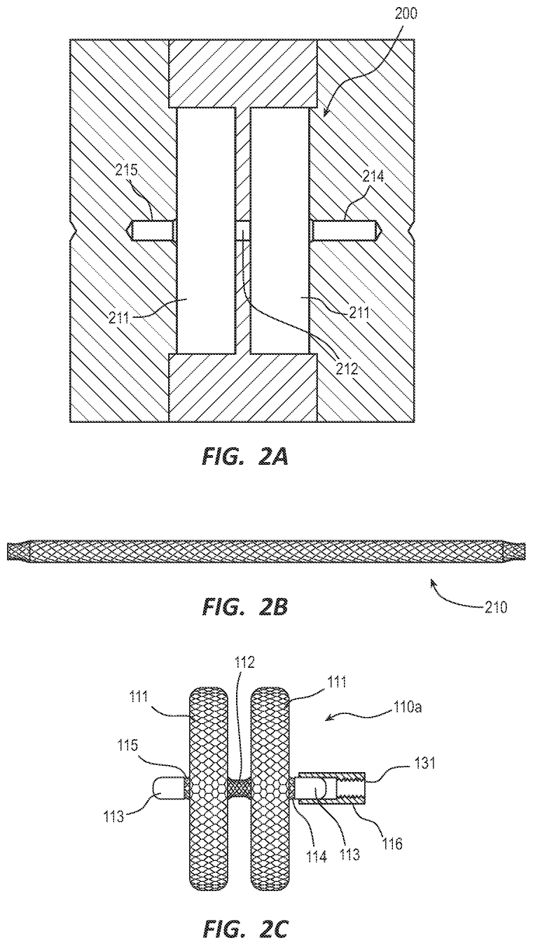

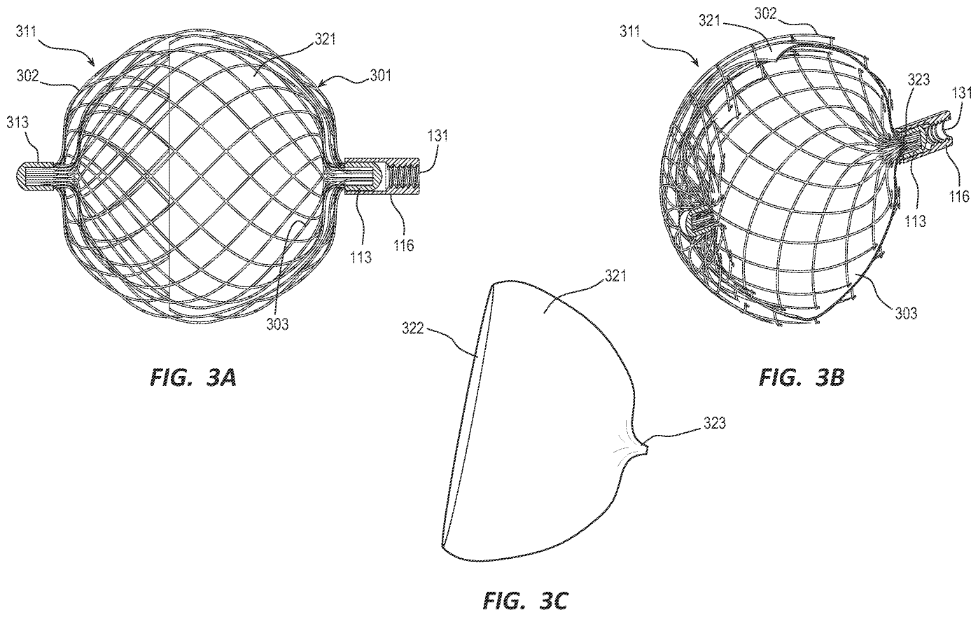

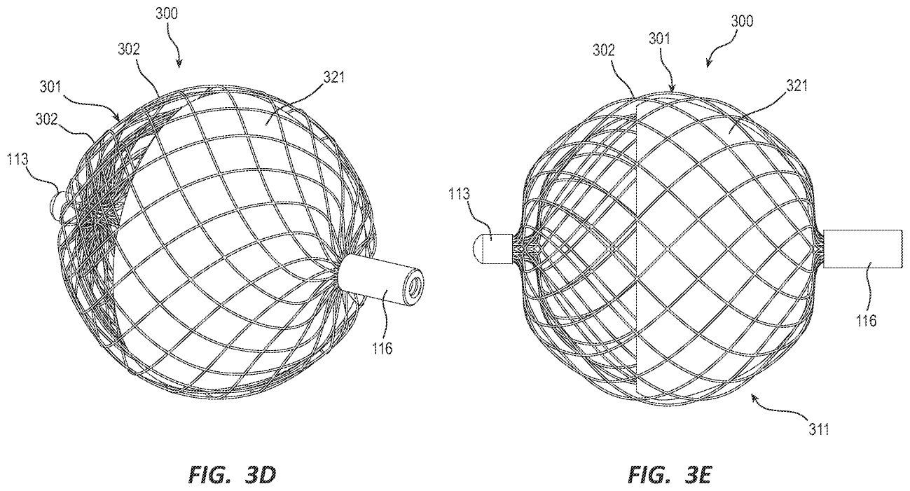

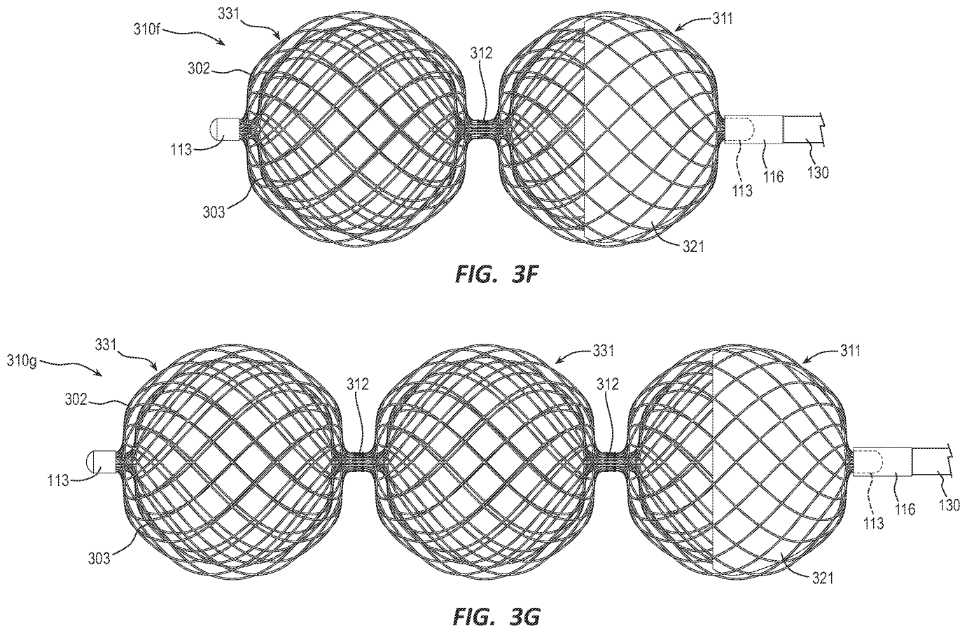

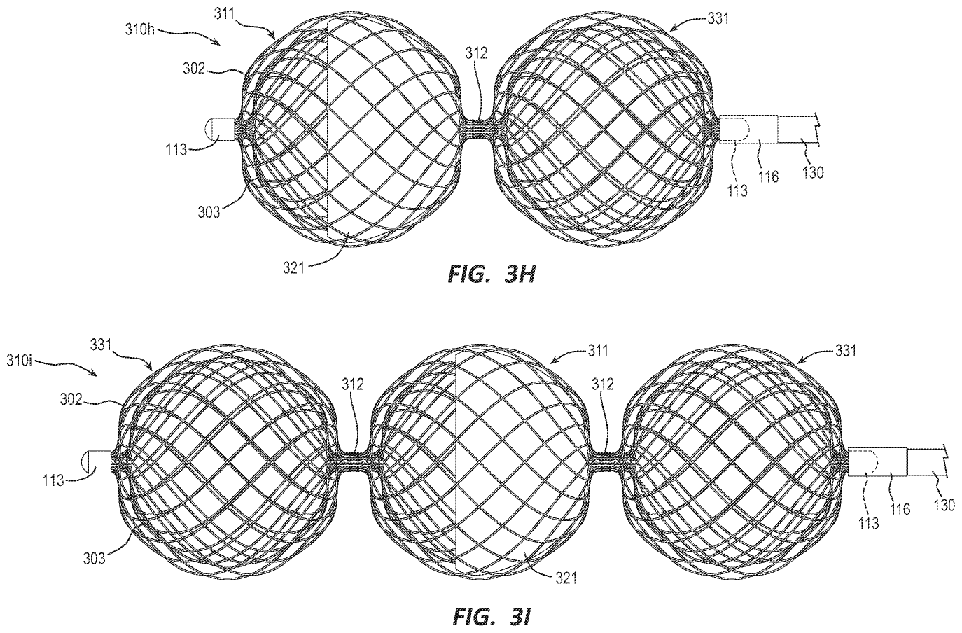

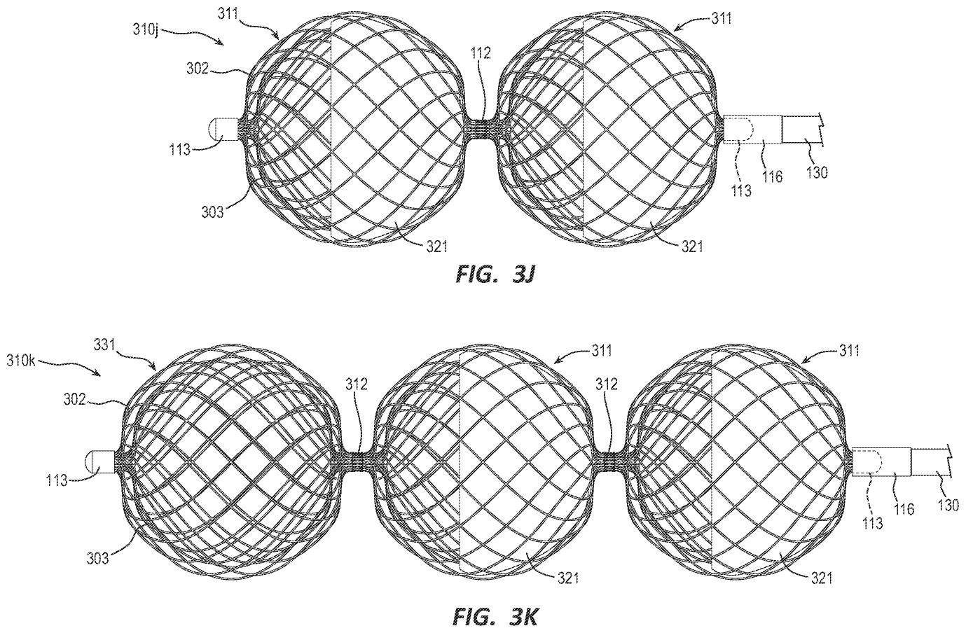

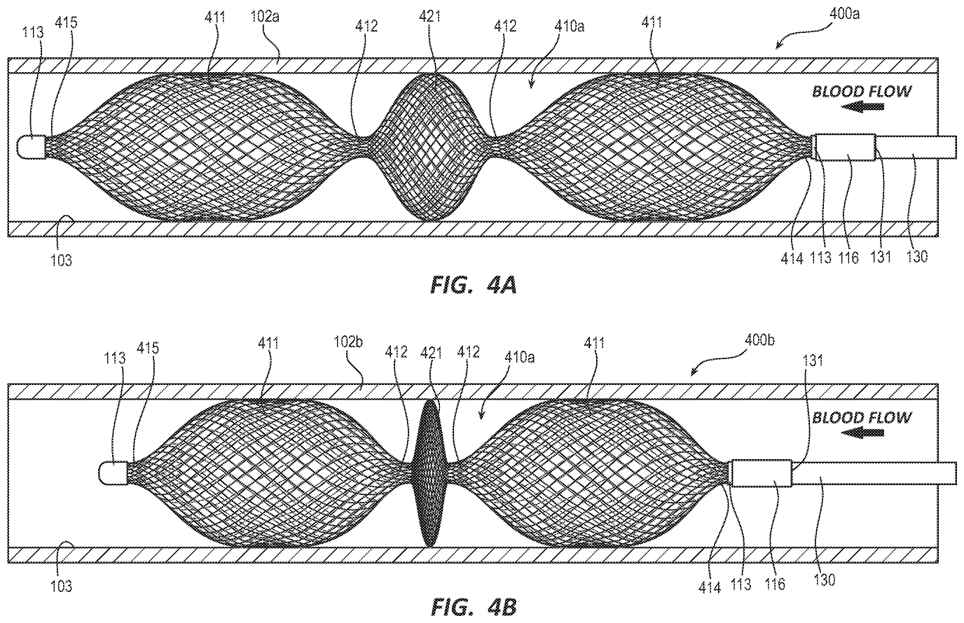

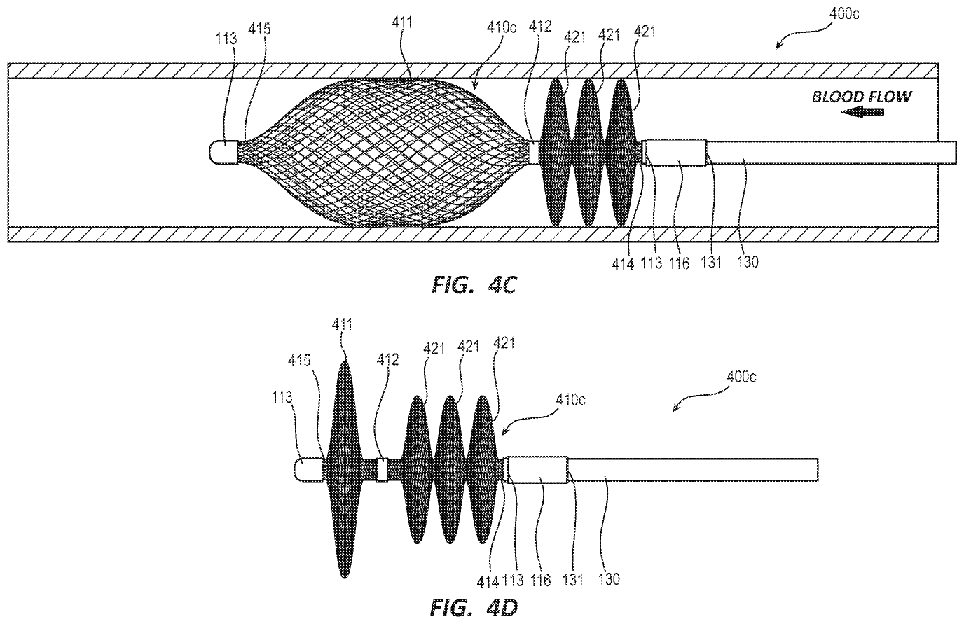

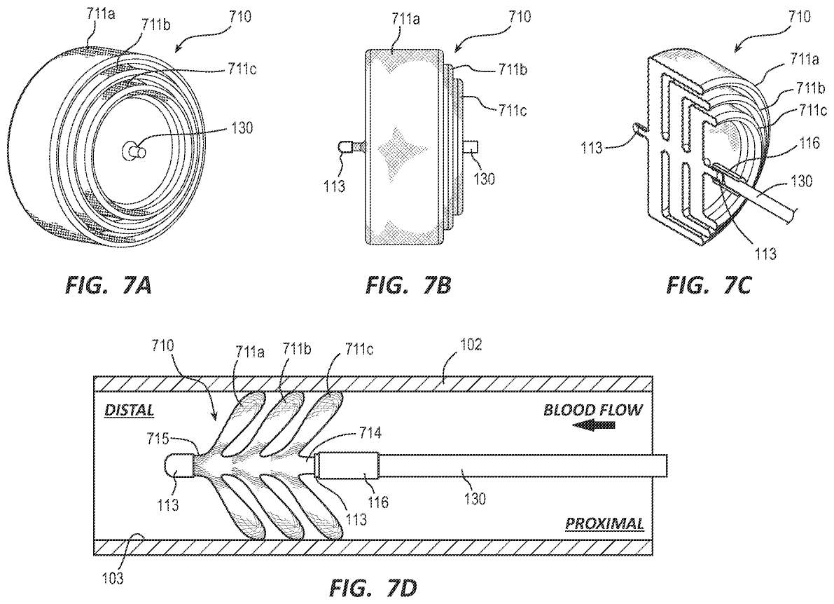

FIGS. 1A-2C illustrate different views of embolization devices including one or more self-expanding enclosures configured to form an elongated shape when deployed, and an exemplary mold for forming the embolization devices, according to an embodiment. FIGS. 3A-3K illustrate different views of embolization devices comprising an instant occluding plug including an inner layer of a braided lattice of nitinol wires, an outer layer of a braided lattice of nitinol wires, and a cup-shaped member disposed between the inner layer and the outer layer of the braided lattices of nitinol wires. FIGS. 4A-6 illustrate different views of embolization devices comprising at least one self-expanding anchor member, one or more occlusion members, and exemplary molds for forming the embolization devices. FIGS. 7A-7D illustrate different views of a self-shortening embolization device including a plurality of enclosures that form an at least partially nested configuration when deployed within a blood vessel. In certain views each device may be coupled to, or shown with, additional components not included in every view. Further, in some views only selected components are illustrated, to provide detail into the relationship of the components. Some components may be shown in multiple views, but not discussed in connection with every view. Disclosure provided in connection with any figure is relevant and applicable to disclosure provided in connection with any other figure or embodiment.

Any of the embolization devices described herein may include a delivery (e.g., placement) wire selectively coupled to the embolic structure of the embolization devices. For example, a delivery wire may include a threaded distal end and the embolic structure of the embolization devices may include a threaded coupling disposed at a proximal end. The delivery wire, then may be configured to threadedly secure to the embolic structure, and the delivery wire may be configured to be rotationally disengaged from the embolic structure.

FIGS. 1A-2C illustrate different views of embolization devices including one or more self-expanding enclosures configured to form an elongated shape when deployed, and an exemplary mold for forming the embolization devices, according to an embodiment. FIG. 1A-1C illustrate different views and/or embodiments of embolization devices in a deployed and expanded state in a blood vessel. FIG. 2A illustrates a cross-sectional view of a mold for forming the embolization device of FIG. 1A, according to an embodiment. FIG. 2B illustrates a braided lattice before being heat set in the mold of FIG. 2A, and FIG. 2C shows illustrates the braided lattice in an expanded state after being heat set in the mold of FIG. 2A.

Turning specifically to FIG. 1A, an embolization device 100a is shown in deployed and at least partially expanded in a blood vessel 102. The embolization device 100a includes an embolic structure 110a comprising a plurality of self-expanding enclosures 111 or baskets. In the illustrated embodiment, the embolic structure 110a includes two self-expanding or baskets 111 with a necked down portion 112 disposed between the enclosures 111. In another embodiment, the embolic structure 110a may include a single enclosure 111 or more than two (e.g., three, four, five, six, etc.) enclosures 111 with necked down portions 112 disposed between each of the more than two enclosures. In certain embodiments, the enclosures 111 of the embolic structure 110a may be all the same size and shape or may be of different sizes and shapes. In some embodiments, when the enclosures 111 number two or more and the shapes are not symmetrical about the placement wire 130, the enclosures 111 may be aligned or misaligned one to another.

In the illustrated embodiment, each of the plurality of self-expanding enclosures include a braided lattice or matrix of braided nitinol wires. The ends of the wires can be restrained by clamps 113 disposed at a proximal end 114 and a distal end 115 to prevent fraying of the braid. In some embodiments, at least one of the clamps 113 may include a radiopaque marker disposed thereon, disposed proximate thereto, and/or integrated therein. For example, a platinum radiopaque marker may be disposed on at least one (e.g. both) of the clamps 113. The embolic structure 110 can be releasably coupled to a placement wire 130 for deployment. For example, in the illustrated embodiment, the embolic structure 110a includes a threaded coupling 116 disposed at the proximal end 114 that can be threadingly coupled to a threaded end 131 of the placement wire 130. When deployed the embolic structure 110 can be rotationally held in place relative to the placement wire 130 when the embolic structure 110 engages with the vessel wall and the placement wire 130 can be rotated to release the placement wire 130 from the embolic structure 110. Other mechanisms for release and deployment are also within the scope of this disclosure including, hooks, collets, loops, snares, and so forth.

The embolization device 100 can be deployed within the blood vessel 102 by advancing the delivery catheter containing the embolic structure 110a to a treatment location in the body and deploying the embolic structure 110a. In some embodiments, this may include loading the delivery catheter containing the constrained embolic structure 110a into a guide catheter and advancing the delivery catheter to a distal end of the guide catheter. The delivery catheter may be displaced proximally relative to the embolic structure 110a such that the embolic structure 110a is disposed within the blood vessel 102. The embolic structure 110a may be configured to self-expand as it is deployed within the blood vessel 102. In some embodiments, the delivery catheter may include a 3 French or smaller delivery catheter.

When deployed within a blood vessel 102, the embolic structure 110a can transition from the constrained state to the at least partially expanded state, such as shown in FIG. 1A. In some embodiments, the enclosures 111 may self-expand when disposed outside the delivery catheter until the enclosures 111 contact a vessel wall 103. The placement wire 130 may be decoupled from the embolic structure 110a, for example by rotating the placement wire relative to the embolic structure 110a to release the placement wire 130 from the embolic structure 110a. As shown, the enclosures 111 of the embolic structure 110a are partially radially expanded.

When deployed, the embolic structure 110a can form a physical blood flow restrictor within the blood vessel 102. Pore size, density of filaments in the enclosures 111, degree of expansion of the enclosures 111, and other parameters may affect the degree to which flow across the device is restricted. Embodiments wherein blood flow is reduced from about 10% to about 50% or to about 100% are within the scope of this disclosure. For example, in some embodiments, blood flow may be reduced from about 10% to about 100%, about 10% to about 25%, about 25% to about 50%, about 50% to about 75%, or about 75% to about 100%. In some embodiments, blood flow may be initially reduced about 10% to about 50% and then about 50% to about 75% or about 75% to about 100% after a predetermined period of time. Each of the enclosures 111 may be sized and dimensioned such that, when deployed within the blood vessel 102, each of the enclosures 111 takes on an elongated, generally cylindrical shape having opposing generally conical, frustoconical, or semispherical end regions.

In the embodiment shown in FIG. 1A, the embolic structure 110a includes two enclosures 111 that take on the elongated, generally cylindrical shape when disposed within the blood vessel 102. Using two enclosures 111 in the embolic structure 110a allows for lower friction on delivery of the embolic structure 110a within the blood vessel 102, while the elongated, generally cylindrical shape taken on by the enclosures 111 when deployed within the blood vessel 102 provides more engagement with the walls 103 of the blood vessel 102, thereby ensuring more apposition to the blood vessel wall 103 and increasing resistance to migration. The elongated enclosures 111 also may increase the likelihood of co-axial deployment within the blood vessel 102.

The elongated shape of the enclosures 111 when deployed also inhibits one or more of the enclosures from tipping or positioning off-center, and allowing leakage if the embolic structure 110a fails. Instead, the if the embolic structure 110a fails, the embolic structure 110a is more likely to migrate through the blood vessel 102 with the flow of blood in the blood vessel 102 than tip over. Migration down the bloodstream maintains occlusion, and is therefore a more desirable failure than tipping of one or more of the enclosures 111, as cross-sectional coverage of the embolized blood vessel 102 is maintained even with migration of the embolic structure 110a. Turning ahead in the drawings to FIG. 1C, the elongated, generally cylindrical shape taken on by the enclosures 111 when deployed within the blood vessel 102 also allows for more easy deployment of the embolic structure 110a in curved blood vessels 102, as the elongated enclosures are less prone to tipping during deployment. Furthermore, if a user attempts to deploy the embolization device 100a with forward pressure (e.g., packing) on the catheter during deployment, the enclosures 111 are less likely to tip sideways within the blood vessel 102. In some embodiments, when deployed in a blood vessel 102 having a diameter of about 3 mm to about 8 mm (e.g., about 3 mm to about 4 mm), each enclosure 111 may include an axial length of about 5 mm to about 8 mm, about 5 mm to about 6 mm, about 6 mm to about 7 mm, about 7 mm to about 8 mm, about 5 mm, about 6 mm, about 7 mm, or about 8 mm. In some embodiments, when deployed in the blood vessel 102, an outer circumferential region of each of the enclosures 111 contacts the blood vessel wall 103. The outer circumferential region of each of the enclosures 111 that contacts the blood vessel wall 103 may include a length of at least about 3 mm, at least about 4 mm, at least about 5 mm, at least about 6 mm, at least about 7 mm, at least about 8 mm, about 3 mm to about 8 mm, about 3 mm to about 4 mm, about 4 mm to about 5 mm, about 5 mm to about 6 mm, about 6 mm to about 7 mm, or about 7 mm to about 8 mm.

When deployed within the blood vessel 102, the enclosures 111 are spaced from one another. For example, the necked down portions 112 may include a length of at least about 0.5 mm, at least about 1.0 mm, at least about 1.5 mm, at least about 2 mm, about 0.5 mm to 2.0 mm, about 0.5 mm to about 1.0 mm, about 1.0 mm to about 1.5 mm, or about 1.5 mm to about 2.0 mm. When deployed, the outer circumferential region of each of the enclosures contacting the blood wall also are spaced from one another, e.g., spaced at least about at least about 2 mm, at least about 3 mm, at least about 4 mm, at least about 5 mm, about 2 mm to about 6 mm, about 2 mm to about 3 mm, about 3 mm to about 4 mm, about 4 mm to about 5 mm, or about 5 mm to about 6 from one another.

Turning now to FIG. 1B, an embolic structure 110b is shown. Unless otherwise noted, the embolic structure 110b may include any aspect of the embolic structure 110a described above. For example, the embolic structure 110b may include one or more (e.g., two) enclosures 111 sized and dimensioned to take on the elongated generally cylindrical shape having opposing generally conical, frustoconical, or semispherical end regions. The embolic structure 110b also may include the clamps 113 disposed at the proximal end 114 and the distal end 115 to prevent fraying of the braid. The embolic structure 110b may include the threaded coupling 116 disposed at the proximal end 114 that can be threadingly coupled to the threaded end 131 of the placement wire 130. The embolic structure 110b may include necked down portions 112 disposed between enclosures 111, 121.

In some embodiments, the embolic structure 110b may include one or more additional self-expanding enclosure being sized and dimensioned to take a shape different than the enclosure(s) 111 when deployed in the blood vessel 102. For example, while the enclosure(s) 111 are sized and dimensioned to take on the elongated generally cylindrical shape having opposing generally conical, frustoconical, or semispherical end regions when deployed in the blood vessel 102, the one or more additional enclosures 121 may be sized and dimensioned to take on a generally spherical shape when deployed within the blood vessel 102. Because the one or more additional enclosures 121 are generally spherical when deployed within the blood vessel 102, less area of the outer periphery circumferentially contacts the blood vessel wall 103 than the enclosures 111. In some embodiments, when deployed in the blood vessel 102, an outer circumferential region of the additional enclosure 121 contacts the blood vessel wall 103. The outer circumferential region of the additional enclosure 121 that contacts the blood vessel wall 103 may include a length of less about 3 mm, less than about 2.5 mm, less than about 2 mm, less than about 1.5 mm, less than about 1 mm, about 1 mm to about 1 mm, about 1 mm to about 2 mm, or about 2 mm to about 3 mm. The additional enclosure 121 also may include an axial length that is less than the axial length of the one or more enclosures 111. For example, each additional enclosure 121 may include an axial length of about 3 mm to about 6 mm, about 3 mm to about 4 mm, about 4 mm to about 5 mm, or about 5 mm to about 6 mm.

Turning to FIG. 2A, a cross-sectional view of a mold 200 for heat setting the embolic structure 110a is illustrated. The mold 200 may include two or more enlarged cavities 211 and multiple channel regions. For example the mold 200 may include a distal channel region 215, a proximal channel region 214, and one or more intermediate channel regions 212 positioned between adjacent enlarged cavities of the two or more enlarged cavities 211. In the embodiment shown in FIG. 2A, the mold 200 includes two enlarged cavities 211 and a single intermediate channel region 212 disposed between the two enlarged cavities 211. In other embodiments, the mold 200 may include three enlarged cavities 211 and two intermediate channel regions 212, each intermediate channel region of the two intermediate channel regions 212 being disposed between different adjacent enlarged cavities of the three enlarged cavities 211. Other embodiments may include additional enlarged cavities and intermediate channel regions.

The channel regions 212, 214, 215 include a channel diameter and the enlarged cavities 211 have a cavity diameter greater than the channel diameter. One or more (e.g., all) of the enlarged cavities 211 may include an axial length (e.g., depth) that is at least about one-eighth the cavity diameter of the enlarged cavities 211, such as at least about one-sixth, at least about one-fifth, at least about one-fourth, at least about one third, about one-eighth, about one-sixth, about one-fifth, about one-fourth, about one-third, about one-eighth to about one-third, about one-eighth to about one-sixth, about one sixth to about one-fourth, or about one-fifth to about one-third the cavity diameter of the enlarged cavities 211. In some embodiments, the diameter of the enlarged cavity may be about 2 mm to about 20 mm, about 2 mm to about 10 mm, about 10 mm to about 20 mm, about 2 mm to about 5 mm, about 5 mm to about 8 mm, about 8 mm to about 11 mm, about 11 mm to about 14 mm, about 14 mm to about 17 mm, or about 17 mm to about 20 mm. In some embodiments, the axial length of the enlarged cavity 211 may be about 0.015 inch to about 0.125 inch, about 0.015 inch to about 0.070 inch, about 0.070 inch to about 0.125 inch, about 0.015 inch to about 0.050 inch, about 0.050 inch to about 0.075 inch, about 0.075 inch to about 0.100 inch, or about 0.100 inch to about 0.125 inch.

In some embodiments, the channels 214, 215 include a diameter of about 0.015 inch to about 0.050 inch, about 0.015 inch to about 0.025 inch, or about 0.025 inch to about 0.050 inch. The channels 214, 215 may include an axial length (e.g., depth) of about 0.025 inch to about 0.125 inch, about 0.025 inch to about 0.075 inch, about 0.075 inch to about 0.125 inch, about 0.025 inch to about 0.050 inch, about 0.050 inch to about 0.075 inch, about 0.075 inch to about 0.100 inch, or about 0.100 inch to about 0.125 inch. In some embodiments, the intermediate channel region 212 may include a diameter of about 0.015 inch to about 0.075 inch, about 0.015 inch to about 0.025 inch, about 0.025 inch to about 0.050 inch, or about 0.050 to about 0.075 inch. The intermediate channel 212 may include an axial length (e.g., depth) of about 0.010 inch to about 0.050 inch, about 0.010 inch to about 0.030 inch, about 0.030 inch to about 0.050 inch, about 0.010 inch to about 0.020 inch, about 0.020 inch to about 0.030 inch, about 0.030 inch to about 0.040 inch, or about 0.040 inch to about 0.050 inch.

FIG. 2B illustrates a braided lattice 210 before being heat set in the mold 200 of FIG. 2A, and FIG. 2C shows illustrates the braided lattice in an expanded state after being heat set in the mold of FIG. 2A. The braided lattice 210 may include a generally tubular structure. Also contemplated herein is a method of forming an embolic structure of an embolization device, such as the embolization structures 110a, 110b. The method may include providing the braided lattice 210 of nitinol wire. The method also may include inserting the braided lattice 210 into the mold 200 having three or more channel regions 212, 214, 215 having a channel diameter and two or more enlarged cavities 211 having a cavity diameter greater than the channel diameter. The method also may include heat setting the braided lattice 210 with two or more portions of the braided lattice 210 disposed within the two or more enlarged cavities 211. Temperatures reached during heat setting, which may vary and be dependent upon materials, are sufficient to effect shape setting for the particular material being shaped in the mold 200.

When inserted into the mold 200, the two or more portions of the braided lattice 210 are more densely disposed within the two or more enlarged cavities 211 than portions of the braided lattice disposed in the three or more channel regions. For example, the braided lattice 210 may be disposed within the two or more enlarged cavities 211 such that the braided lattice is fully packed within the respective cavity 211 and cannot be axially compressed any further within the respective enlarged cavity 211 (similar to the solid height of a completely compressed spring). Conventional cavities having less axial length or depth typically do not allow for this axial compression of the braided lattice within the cavities. Instead, when disposed within the cavity, the braided lattice would undesirably fold within the cavity because the bending of the wire in the braided lattice takes up too much space before folding back on itself. The axial depth of the enlarged cavities 211, however, provide the depth necessary to prevent this and allow for more uniform packing and compression of the braided lattice 210 in the enlarged cavities 211 before heat setting.

FIG. 2C illustrates the braided lattice 210 after heat setting in the mold 200 and with the clamp 113 threaded coupling 116 secured thereto to form the embolic structure 110a. In the illustrated embodiment, in the pre-load or expanded state of the embolic structure 110a, the enclosures 111 have a wider disk shape. In certain embodiments, the embolic structure 110a may be provided to a user in the expanded state such as shown in FIG. 2C. When preparing the embolic structure 110a for use, the user may transition the embolic structure 110a into a constrained state by pulling or otherwise disposing the embolic structure 110a into a delivery catheter to reduce a diameter of the embolic structure 110a. In another embodiment, the embolic structure 110a may be provided to a user in the constrained state where the embolic structure 110a are crimped to a small diameter and disposed within the delivery catheter.

As provided above, the embolization device 100a can be deployed within a blood vessel by advancing the delivery catheter containing the embolic structure 110a to a treatment location in the body and deploying the embolic structure 110a. The embolic structure 110b or any other embolic structure described herein may be similarly deployed as described in relation to the embolic structure 110a. Also contemplated herein is a method for restricting blood flow within the blood vessel 102. The method may include loading the delivery catheter containing the constrained embolic structure 110a into a guide catheter and advancing the delivery catheter to a distal end of the guide catheter. The method also may include positioning an embolization device, such as the embolization device 100a or 100b into the blood vessel 102 adjacent to a treatment site. Accordingly, the method also may include deploying the embolization device from a delivery catheter into the blood vessel 102 at the treatment site.

In some embodiments, the method for restricting blood flow within the blood vessel may include self-expanding a plurality of self-expanding enclosures of the embolic structure 110a or 110b of the embolization device within the blood vessel 102. The plurality of self-expanding enclosures contact a wall 103 of the blood vessel 102, and at least one of the plurality of self-expanding enclosures 111 self-expands to an elongated generally cylindrical shape having opposing generally conical, frustoconical, or semispherical end regions. The method also includes restricting blood flow through the embolic structure 110a, 110b. The blood vessel may include a diameter of about 1.5 mm to about 8 mm, and an outer circumferential region of the at least one of the plurality of self-expanding enclosures that contacts the blood vessel may have a length of about 3 mm to about 8 mm contacting the wall 103 of the blood vessel 102. The plurality of self-expanding enclosures may be spaced at least 2 mm from one another when deployed within the blood vessel 102.

In some embodiments, the method for restricting blood flow within the blood vessel 102 may include no more than two self-expanding enclosures 111, and each of the two self-expanding enclosures 111 self-expand to the elongated generally cylindrical shape having opposing generally conical, frustoconical, or semispherical end regions. In some embodiments, the plurality of self-expanding enclosures include self-expanding enclosures sized and dimensioned to form different shapes when deployed within the blood vessel 102. For example, in some embodiments of the method for restricting blood flow within the blood vessel 102, the plurality of self-expanding enclosures include two self-expanding enclosures 111 and an additional self-expanding enclosure 121. The two self-expanding enclosures 111 self-expand in the blood vessel 102 to the elongated generally cylindrical shape having opposing generally conical, frustoconical, or semispherical end regions. The additional self-expanding enclosure 121 is disposed between the two self-expanding enclosures 121 and self-expands to a generally spherical shape.

When deployed, the embolic structure 110 can form a physical blood flow restrictor within the blood vessel 102. Pore size, density of filaments in the enclosures 111, degree of expansion of the enclosures 111, and other parameters may affect the degree to which flow across the device is restricted. Embodiments wherein blood flow is reduced from about 10% to about 100% are within the scope of this disclosure. In some embodiment, blood flow may be reduced from about 10% to about 100%, about 10% to about 25%, about 25% to about 50%, about 50% to about 75%, or about 75% to about 100%. In some embodiments, blood flow may be initially reduced about 10% to about 50% and then about 50% to about 75% or about 75% to about 100% after a predetermined period of time. The placement wire 130 may be decoupled from the embolic structure 110, for example by rotating the placement wire relative to the embolic structure 110 to release the placement wire 130 from the embolic structure 110.

Turning now to FIGS. 3A-3K, embodiments of embolization devices according to this disclosure may include an instant occluding plug. Generally, some embodiments of an embolization device may include an inner layer of a braided lattice of nitinol wires, an outer layer of a braided lattice of nitinol wires, and a cup-shaped member disposed between the inner layer and the outer layer of the braided lattices of nitinol wires. The cup-shaped member is effective to provide instant occlusion when deployed within a blood vessel. Unless otherwise noted, embolization devices shown in or relating to FIGS. 3A-3K may include any aspect of the embolization devices 100a, 100b, such as systems and methods for delivering and/or deploying the embolization devices into a blood vessel 102 to effect a slowing and/or cessation of blood flow.

Turning to FIGS. 3A and B, cross-sectional views of a braided lattice 301 of nitinol wires and a cup-shaped member 321 are illustrated. Unless otherwise noted, the braided lattice 301 of nitinol wires may include any aspect of the braided lattice of nitinol wires of the enclosures 111, 121 provided above. In many embodiments, the braided lattice 301 of nitinol wires includes an outer layer 302 and an inner layer 303. The ends of the wires can be restrained by clamps 113 disposed at a proximal end 314 and a distal end 315 of the braided lattice 301 to prevent fraying of the braid. In some embodiments, at least one of the clamps 113 may include a radiopaque marker disposed thereon, disposed proximate thereto, and/or integrated therein. For example, a platinum radiopaque marker may be disposed on at least one (e.g. both) of the clamps 113. The braided lattice 301 can be releasably coupled to a placement wire 130 for deployment. For example, in the illustrated embodiment the threaded lattice 301 includes a threaded coupling 116 disposed at the proximal end 314 that can be threadingly coupled to a threaded end 131 of the placement wire 130.

Turning now to FIG. 3C, a cup-shaped member 321 is illustrated. The cup-shaped member includes an open end 322 and may include pointed tip 323 opposite to the open end 322. In some embodiments, the cup-shaped member 321 is generally semispherical. More particularly, the cup-shaped member 321 may be shaped to include at least half of a generally spherical shape. In some embodiments, the cup-shaped member 321 is shaped to include about one-half to about three-quarters of a generally spherical shape. The cup-shaped member 321 may include one or more of various materials. For example, the cup-shaped member 321 may include one or more of expanded polytetrafluoroethylene, fabric, polyester, nylon, and/or polyether block amide. The cup-shaped member 321 may be formed as a thin layer, such as 0.0005 inch to about 0.010 inch, less than about 0.010 inch, less than about 0.008 inch, less than about 0.006 inch, less than about 0.002, or less than about 0.001 inches.

Turning now to FIGS. 3D-3E, an embolic structure 300 comprising an enclosure 311 including the braided lattice 301 and the cup-shaped member 321 is illustrated. The cup-shaped member 321 is disposed in the enclosure 311 between the inner layer 303 and the outer layer 302 of the braided lattice 301 of nitinol wires, according to an embodiment. In some embodiments, the pointed tip 323 may be at least partially disposed in the clamp 113. In some embodiments, the pointed tip 323 includes an aperture and the inner layer 303 of the braided lattice 301 passes through the aperture of the pointed to 323 to an additional enclosure or the clamp 113. When disposed within the braided lattice 301, the cup-shaped member extends at least halfway from the along the braided lattice. For example, the pointed end 323 of the cup-shaped member 321 may be disposed at a first or proximal end 314 of the braided lattice 301 and may extend, between the inner layer 303 and the outer layer 302, at least halfway to a second or distal end 315 of the braided lattice 301. In some embodiments, the braided lattice 301 is generally spherical, and the cup-shaped member 321 extends about one-half to about three-quarters of the generally spherical shape of the braided lattice 301. Extending the cup-shaped member 321 beyond a midpoint between the ends of the braided lattice 301 inhibits the cup-shaped member 321 from peeling back during use as a result of blood flow or blood pressure, regardless of the direction of blood flow. The cup-shaped member 321, however, does not form a complete sphere, so as to prevent a place for an air bubble to form in the blood vessel.

In some embodiments, the embolic structure 300 includes only a single enclosure 311. In other embodiments, however, embolic structures according to this disclosure may include multiple enclosures with at least one or more enclosures including the cup-shaped member 321 and one or more enclosures 331 having no cup-shaped member 321. Multiple enclosures are beneficial for conforming around bends in blood vessels. FIGS. 3F-3K illustrate various embodiments of embolic structures 310a-f having various combinations of enclosures 331 with no cup-shaped member 321 and enclosures including the cup-shaped member 321.

Turning specifically to FIG. 3F, an embolic structure 310f may include a plurality of enclosures, according to an embodiment. The plurality of enclosures in the embolic structure 310f may include a single first enclosure 311 having the cup-shaped member 321 disposed between the inner layer 303 and the outer layer 302 of the braided lattice of nitinol. The plurality of enclosures in the embolic structure 310f also may include a single second enclosure 311 having no cup-shaped member disposed between the inner layer 303 and the outer layer 302 of the braided lattice 301. In some embodiments, a necked down portion 312 is disposed between the first enclosure 311 and the second enclosure 331. The outer layer 302 and the inner layer 303 may extend continuously through the necked down portion 312 between the first enclosure 311 and the second enclosure 331, according to an embodiment. In some embodiments, only one of the outer layer 302 and the inner layer 303 extends continuously through the necked down portion 312 between the first enclosure 311 and the second enclosure 331, according to an embodiment. In some embodiments of the enclosure 331, the inner layer 303 of the braided lattice is absent, and the enclosure 331 may include any enclosure of a braided lattice described herein. In some embodiments, each enclosure 311, 331 includes a clamp 113 at both ends of the respective enclosure 311, 331 that secures the inner layer 303 and the outer layer 302 of each enclosure 311, 331 together. Clamps 113 of adjacent enclosures 311, 331 may be secured together with a coupling.

In the illustrated embodiment of the embolic structure 310f, the embolic structure 310f includes only two enclosures 311, 331, and the opening 322 of the cup-shaped member 321 in the enclosure 311 is oriented towards the enclosure 331 having no cup-shaped member 321. In other embodiments, embolic structures may include different numbers of enclosures and orientations of the cup-shaped member 321. Turning now to FIG. 3G, an embolic structure 310g may include one enclosure 311 including the cup-shaped member 321 and two enclosures 331 having no cup-shaped members. The opening 322 of the cup-shaped member 321 in the enclosure 311 is oriented towards both of the two enclosures 331 having no cup-shaped member 321.

Turning now to FIG. 3H, an embolic structure 310h may include only one enclosure 311 having the cup-shaped member 321 and only one enclosure 331 having no cup-shaped member 321. In the embolic structure 310h, however, the opening 322 in the cup-shaped member 321 of the enclosure 311 may be oriented away from the enclosure 331 having no cup-shaped member 321.

Turning now to FIG. 3I, an embolic structure 310i may include only one enclosure 311 having the cup-shaped member 321 and only two enclosures 331 having no cup-shaped member 321. The one enclosure 311 including the cup-shaped member 321 may be disposed between the two enclosures 331 having no cup-shaped member 321. Accordingly, the opening 322 in the cup-shaped member 321 of the enclosure 311 may be oriented away from a first enclosure 331 having no-cup-shaped member 321 and oriented towards a second enclosure 331 having no cup-shaped member.

Turning now to FIG. 3J, an embolic structure 310j may include only two enclosures 311, with both enclosures 311 including the cup-shaped member 321. Accordingly, the opening 322 of one cup-shaped member 321 is oriented towards one of the two enclosures 311, and the opening 322 of the other cup-shaped member 321 is oriented away from the other of the two enclosures 311. Enclosures 331 having no cup-shaped member 321 may be absent from the embolic structure 310j.

Turning now to FIG. 3K, an embolic structure 310k may include only two enclosures 311 including the cup-shaped member 321 and only one enclosure 331 having no cup-shaped member 331. The enclosures 311, 331 may be disposed such the opening 322 of the cup-shaped member 321 of both of the two enclosures 311 is orientated towards the enclosure 331 having no cup-shaped member 321. Accordingly, the opening 322 of the cup-shaped member 321 of a first enclosure 311 may be oriented towards the enclosure 331 but away from a second enclosure 311, and the opening 322 of the cup-shaped member 321 of the second enclosure 311 may be oriented towards both the enclosure 331 and the first enclosure 311.

Also contemplated herein is a method for restricting blood flow within a blood vessel using any of the embodiments of embolic structures 310, 310f-k described above. Unless otherwise noted, methods of restricting blood flow using any of the embodiments of embolic structures 310, 310f-k may include any aspect of the methods of restricting blood flow using the embolic structures 110a, 110b described above. In some embodiments, the method for restricting blood flow within a blood vessel includes positioning an embolization device into the blood vessel adjacent to a treatment site, deploying the embolization device from a delivery catheter into the blood vessel at the treatment site, and disposing at least one enclosure of an embolic structure of the embolization device within the blood vessel. The at least one enclosure may include the enclosure 311 including the cup-shaped member 321 disposed between the inner layer 303 and the outer layer 303 of the braided lattice 301 of nitinol wire. The opening 322 of the cup-shaped member 321 may be oriented away from a direction of blood flow in the blood vessel when the embolic structure is disposed in the blood vessel. Any of the embolic structures 310, 310f-k may be similarly disposed in the blood vessel in the method for restricting blood flow within the blood vessel.

Also contemplated herein are embolic structures including both an anchor member and an occlusion member. FIGS. 4A-6 illustrate different views of embolization devices including embolic structures comprising at least one self-expanding anchor member and one or more occlusion members, and exemplary molds for forming the embolization devices. In some instances the landing zone for deploying an embolization device is too short to accommodate the length of the embolization device. To compensate, embolization devices with an embolic structure having a more stiff anchor member that elongates during implantation/deployment of the embolization device and a less stiff occlusion member that does not substantially elongated after implantation/deployment of the embolization device. When not restricted within a blood vessel, the anchor member may self-expand to an enclosure having a larger diameter than the occlusion member. Furthermore, while both the anchor member and the occlusion member may include a braided lattice of nitinol wire, the nitinol wire may be more densely disposed in the braided lattice of the occlusion member than the anchor member. Various embodiments described herein may include different numbers of anchor members and/or occlusion members in each embolization device. Unless otherwise noted, delivery and deployment of the embolization devices including an anchor member and an occlusion member may be substantially the same or similar to delivery and deployment of other embolization devices and structures described herein.

Turning specifically to FIGS. 4A and 4B, an embolization device 400a is shown in deployed and expanded in a more narrow (e.g., 5 mm) blood vessel 102a and a more wide (e.g., 6 mm) blood vessel 102b. The embolization device 400a includes an embolic structure 410a comprising a plurality of self-expanding enclosures or baskets comprising a braided lattice of nitinol wire. The plurality of self-expanding enclosures include two anchor members 411 and an occlusion member 421 disposed between the two anchor members 411, with necked down portions disposed between the plurality of self-expanding enclosures. In other embodiments, the embolic structures may include other numbers and placement of anchor members 411 and occlusion member 421.

In the illustrated embodiment, each of the plurality of self-expanding enclosures (e.g., the anchor member 411 and the occlusion member 421) include a braided lattice or matrix of braided nitinol wires. The ends of the wires can be restrained by clamps 113 disposed at a proximal end 414 and a distal end 415 to prevent fraying of the braid. That is, embodiments wherein each self-expanding enclosure (e.g. anchor member 411 and occlusion member 421) are formed from continuous wires that run from a proximal end of the embolization device 400a to a distal end of the embolization device 400a are within the scope this disclosure. The wires may be continuous from a proximal clamp 113 to a distal clamp 113.

In some embodiments, the embolic structure 410a may also include clamps between and/or connecting adjacent anchor members 411 and/or occlusion members 421. In some embodiments utilizing continuous wires, each wire of the braid may pass through the clamps 412, while still forming the embolization device 400a from a continuous braid. In such embodiments, the clamps 412 may be configured to isolate the portions of the embolization device 400a from each other, such that a load applied to the wires of one portion (such as an occlusion member 421) is not directly transferring along the continuous wires to the other portions, but first interacts with the clamp 412 between the members. These clamps 412 may, for example, constrain the wires of the braid to each other at the clamp 412, potentially transferring or equalizing loads on various wires. Additionally, embodiments wherein the embolic structure 410a does not include clamps 412, and only includes necked down or smaller diameter segments at these locations are also within the scope of this disclosure.

Additionally, embodiments wherein one or more portions (e.g. anchor member 411 and occlusion member 421) are formed from different wires are also within the scope of this disclosure. That is, one or more portions may be comprised of wires or braids that terminate at the claims 412. For example, as described in greater detail below, the wire in the braided lattice of the anchor member 411 may be different than wire of the braided lattice of the occlusion member 421, such that the wire in the braided lattice of the plurality of enclosures is not continuous. A clamp 412 or other coupling, then, may connect adjacent anchor members 411 and occlusion members 421. The clamp 412 is configured to secure to and/or connect adjacent anchor members 411 and occlusion members 421 include any aspect of the clamps 113. For example, the clamps 412 may include a radiopaque marker secured thereto and/or incorporated therein. The clamp 412 may help isolate the occlusion member 421 and the adjacent anchor member 411, and may simplify manufacture of the embolic structure 410. This isolation between adjacent members 411, 412 also may allow for more stiff embolic structures 410. In some embodiments, an anchor member 411 disposed adjacent to an occlusion member 421 may be connected with one or more of welding, adhesive (e.g., gluing), threading, or crimping.

In some embodiments, at least one of the clamps 113 may include a radiopaque marker disposed thereon, disposed proximate thereto, and/or integrated therein. For example, a platinum radiopaque marker may be disposed on at least one (e.g. both) of the clamps 113. The embolic structure 410a can be releasably coupled to a placement wire 130 for deployment. In the illustrated embodiment the embolic structure 410a includes a threaded coupling 116 disposed at the proximal end 414 that can be threadingly coupled to a threaded end 131 of the placement wire 130. When deployed the embolic structure 410a can be rotationally held in place relative to the placement wire 130 when the embolic structure 410a engages with the vessel wall and the placement wire 130 can be rotated to release the placement wire 130 from the embolic structure 410a. Other mechanisms for release and deployment are also within the scope of this disclosure including, hooks, collets, loops, snares, and so forth. The embolization device 400a can be deployed within the blood vessel 102 as described above in relation to the embolization device 100a.

When deployed within a blood vessel 102a, 102b, the embolic structure 410a can transition from the constrained state to the expanded state, such as shown in FIGS. 4A and 4B. In some embodiments, the enclosures 411, 421 may self-expand when disposed outside the delivery catheter until the enclosures 411, 421 contact the vessel wall 103. When deployed, the embolic structure 410a can form a physical blood flow restrictor within the blood vessel 102. Pore size, density of filaments in the enclosures 411, 421 degree of expansion of the enclosures 411, 421 and other parameters may affect the degree to which flow across the device is restricted. Embodiments wherein blood flow is reduced from about 10% to about 50% or more are within the scope of this disclosure. In some embodiment, blood flow may be reduced from about 10% to about 100%, about 10% to about 25%, about 25% to about 50%, about 50% to about 75%, or about 75% to about 100%. In some embodiments, blood flow may be initially reduced about 10% to about 50% and then about 50% to about 75% or about 75% to about 100% after a predetermined period of time.

Each of the anchor members 411 may be sized and dimensioned such that, when deployed within either the more narrow blood vessel 102a the more wide blood vessel 102b, each of the anchor members 411 takes on an elongated, generally cylindrical shape having opposing generally conical, frustoconical, or semispherical end regions. In the embodiment shown in FIGS. 4A and 4B, the embolic structure 410a includes two anchor members that take on the elongated, generally cylindrical shape when disposed within the blood vessel 102. The anchor members 411 having an elongated, generally cylindrical shape may provide any of the advantages and/or dimensions described above in relation to the self-expanding enclosures 111.

Each of the occlusion members 421 may be sized and dimensioned such that, when deployed within the blood vessel 102, each of the occlusion members 421 takes on a generally spherical, shortened ellipsoid, or disk shape. In some embodiments, the shape of the occlusion member 421 may vary according to the diameter of the blood vessel. For example, the occlusion member 421 may take on a generally spherical or shortened ellipsoid shape in the more narrow blood vessel 102a (shown in FIG. 4A), but may take on a generally disk shape in a more wide blood vessel 102b (shown in FIG. 4B). In any event, while both the occlusion member 421 and the anchor member 411 may be generally disk-shaped when in an unconstrained state (e.g., not constrained within a blood vessel), the diameter of the occlusion member 421 when in the unconstrained state is smaller than the diameter of the anchor member 411 when in the unconstrained state.

The braided lattice of the anchor member 411 may include nitinol wire that is more stiff but braided less densely than the nitinol wire of the braided lattice of the occlusion member 421. Accordingly, the braided lattice of the occlusion member 421 may include nitinol wire that is less stiff (e.g., more soft) but braided more densely than the nitinol wire of the braided lattice of the anchor member 411. For example, the anchor member 411 may include a braided lattice of nitinol wire braided at a first picks per unit, while the occlusion member 421 may include a braided lattice of nitinol wire that is braided more densely than the nitinol wire of the anchor member 411. The anchor member 411, having more radial force than the occlusion member 421, may then serve as an anchor to resist migration of the embolic structure 410a and provide stability in the blood vessel 102a, 102b.

In some embodiments, the nitinol wire of the braided lattice of the anchor member 411 includes a diameter and the nitinol wire of the braided lattice of the occlusion member 421 includes a diameter that is less than the diameter of the nitinol wire of the anchor member 411. For example, the diameter of the nitinol wire of the braided lattice of the anchor member 411 may be about 0.0015 inches to about 0.0025 inches, and the diameter of the nitinol wire of the braided lattice of the occlusion member 421 may be about 0.0008 inches to about 0.00125 inches. In some embodiments, the braided lattice of the anchor member 411 includes fewer nitinol wires braided together than braided together in the occlusion member 421. For example, the braided lattice of the anchor member 411 may include about 16 nitinol wires to about 72 nitinol wires braided together, and the braided lattice of the occlusion member 421 may include about 64 nitinol wires to about 72 nitinol wires braided together.

In some embodiments, the braided lattice of the anchor member 411 may include various predetermined combinations of a particular number of nitinol wires having a particular diameter. For example, the anchor member 411 may include about 16 wires having a diameter of about 0.0025 inches braided together, about 32 wires having a diameter of about 0.002 inches braided together, about 64 wires having a diameter of about 0.00175 braided together, or about 72 wires having a diameter of about 0.0015 braided together.

In some embodiments, the braided lattice of the occlusion member 421 may include various predetermined combinations of a particular number of nitinol wires having a particular diameter. For example, the occlusion member 421 may include about 64 wires having a diameter of about 0.0008 inches, about 0.001 inches, or about 0.00125 inches. In some embodiments, the braided lattice of the occlusion member 421 may include about 72 wires having a diameter of about 0.0008 inches, about 0.001 inches, or about 0.00125 inches.

Embodiments of embolic structures may include various combinations of anchor members 411 and occlusion members 421. Turning to FIGS. 4C and 4D, an embolization device 400c may include an embolic structure 410c having one distally positioned anchor member 411 and multiple (e.g., three) proximal occlusion members 421. FIG. 4C shows the embolization device 400c deployed in the blood vessel 102, and FIG. 4D illustrates the embolization device in an unconstrained state before being deployed in the blood vessel 102. In the unconstrained state illustrated in FIG. 4D, the diameter of the anchor member 411 is greater than the diameter of each of the occlusion members 421. Other embodiments may include other numbers of anchor members 411 and occlusion members 421. For example, the embolic structure 410c may include: one distal anchor member 411 and one proximal occlusion member 421; one distal anchor member 411 and two proximal occlusion members 421; two distal anchor members 411 and one proximal occlusion member 421; two distal anchor members 411 and two proximal occlusion member 421; two distal anchor members 411 and three proximal occlusion members 421; or one distal occlusion member 421 and two proximal anchor members 411.

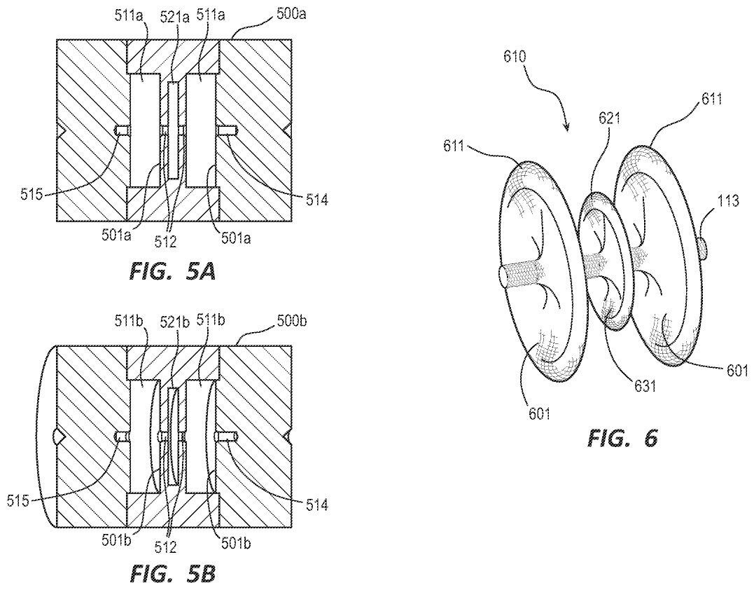

Turning ahead in the drawings to FIG. 5A, a cross-sectional view of a mold 500a for heat setting an embolic structure 410a is illustrated. The mold 500a may include one or more (e.g., two) enlarged anchor cavities 511a, one or more (e.g., one) enlarged occlusion cavities 521a, and multiple channel regions. Other embodiments of the mold 500a may include any number of enlarged anchor cavities 511a and enlarged occlusion cavities 521a complementary to embodiments of embolic structures described above. The enlarged anchor cavities 511a have a volume greater than the volume of the enlarged occlusion cavity 521a, and may include any aspect of the enlarged cavities 211 described above. The mold 500 may include a distal channel region 515, a proximal channel region 514, and one or more (e.g., two) intermediate channel regions 512 positioned between adjacent enlarged cavities 511a, 521a.

The channel regions 512, 514, 515 include a channel diameter and the enlarged cavities 511a, 521a have a cavity diameter greater than the channel diameter. One or more (e.g., all) of the enlarged anchor cavities 511a may include an axial length that is at least about one-eighth the cavity diameter of the enlarged anchor cavities 511a, such as at least about one-sixth, at least about one-fifth, at least about one-fourth, at least about one third, about one-eighth, about one-sixth, about one-fifth, about one-fourth, about one-third, about one-eighth to about one-third, about one-eighth to about one-sixth, about one sixth to about one-fourth, about one-fifth to about one-third the cavity diameter of the enlarged anchor cavities 511a. In some embodiments, each of the enlarged anchor cavities 511a may include any dimensions of the enlarged cavity 211 provided above. Each of the enlarged anchor cavities 511a may include generally planar or flat walls, including a generally planar proximal wall 501a. The enlarged occlusion cavity 521a may include a diameter smaller than the diameter of each of the enlarged anchor cavities 511a. The enlarged cavity 521a also may include an axial length that is less than the axial length of each of the enlarged anchor cavities 511a. For example, the axial length of each of the enlarged anchor cavities 511a may be at least two times, at least three times, at least four times, or at least five times the axial length of the enlarged occlusion cavity 521a.

A braided lattice may be inserted into the mold 500a and heat set to form the embolic structure 410a. The braided lattice inserted into the mold 500a may be generally tubular and may include two anchor regions having the various wire diameters and/or braiding densities described above in relation to the anchor member 411. The braided latticed inserted into the mold 500a also may include an occlusion region having the various wire diameters and/or braiding densities described above in relation to the occlusion member 421.

Accordingly, a method of forming an embolic structure of an embolization device also is contemplated herein. The method may include providing a braided lattice of nitinol wire having at least one anchor region braided at a first picks per unit and one or more occlusion regions braided at a second picks per unit greater than the first picks per unit such that the one or more occlusion regions are braided more densely than the at least one anchor region. The method also may include inserting the braided lattice into the mold 500a such that the at least one anchor region is disposed in at least one enlarged anchor cavity 511a and the one or more occlusion regions are disposed in one or more enlarged occlusion cavities 521a. The method also may include heat setting the braided lattice with the at least one anchor region disposed in the at least one enlarged anchor cavity to form at least one anchor member 411 and the one or more occlusion regions disposed in the one or more enlarged occlusion cavities 521a to form one or more occlusion members 421.

In some embodiments, an embolic structure may be formed to have a concave region on at least one of the anchor member or the occlusion member. FIG. 6, for example, illustrated an embolic structure 610 including anchor members 611 having a concave region 601 and an occlusion member 621 having a concave region 631. FIG. 5B illustrates a mold 500b that may be used to form the embolic structure 610. Unless otherwise noted, the anchor member 611 may include any aspect of the anchor member 411 described above, and the occlusion member 621 may include any aspect of the occlusion member 421 described above. Similarly, unless otherwise noted, the mold 500b may include any aspect of the mold 500a. For example, the mold 500b may include one or more (e.g., two) enlarged anchor cavities 511b, and one or more enlarged occlusion cavities 521b. The mold 500b may include a convex wall 501b defining a portion of the enlarged anchor cavity 511b such that the enlarged anchor cavity 511b includes a concave region. The mold 500b also may include a convex wall defining a portion of the enlarged occlusion cavity 521b such that the enlarged occlusion cavity 521b includes a concave region. When a braided lattice is heat set in the mold 500b (as described above in relation to the mold 500a) the convex walls 501b in the enlarged anchor cavity 511b and the enlarged occlusion cavity 521b result in a concave region 601 being formed in the anchor member 611 and a concave region 631 being formed in the occlusion member 621. At least one (e.g., both) of the concave regions 601, 631 may result in the embolic structure 610 having a decrease in length than conventional embolic devices, while also simultaneously providing the embolic structure 610 with increased radial force. The embolic structure 610 also including the concave regions 601, 631 also may allow the user to more easily and efficiently adjust the embolic structure 610 by pushing the embolic structure. For example, a doctor deploying or adjusting the embolic structure 610 may push on the embolic structure 610 to compress the embolic structure longitudinally. The concave regions 601, 631 may be configured to allow the embolic structure to more easily compress and/or fold.

Also contemplated herein is a method of restricting blood flow within a blood vessel using any of the embolic structures having one or more anchor members and one or more occlusion members described herein. The method may include positioning an embolization device into the blood vessel adjacent to a treatment site. The method also may include deploying the embolization device from a delivery catheter into the blood vessel at the treatment site to dispose at least one self-expanding anchor member and one or more occlusion members of an embolic structure in the blood vessel. The at least one self-expanding anchor member may comprise a braided lattice of nitinol wire braided at a first picks per unit and the one or more occlusion members comprising a braided lattice of nitinol wire braided at a second picks per unit greater than the first picks per unit such that the nitinol wire of the occlusion portion is braided more densely than the nitinol wire of the anchor portion. The method also may include self-expanding the at least one self-expanding anchor member to an elongated generally cylindrical shape that contacts a wall of the blood vessel, and restricting blood flow through the embolic structure.

Turning now to FIGS. 7A-7D, in some embodiments, an embolization device may be configured to at least partially nest within itself when deployed within a blood vessel. For example, a self-shortening embolization device may include a plurality of enclosures comprising a braided lattice that form an at least partially nested configuration when deployed within a blood vessel. The at least partially nesting configuration of the embolization device achieves a shorter implant length, while still having a desired density of braids in the plurality of enclosures to provide the desired reduction of blood flow in the blood vessel.

FIGS. 7A-7C illustrate various views of an embolic structure 710 having a plurality of enclosures 711a-c in a manufactured state, and FIG. 7D illustrates the embolic structure 710 deployed in the blood vessel 102. The plurality of enclosures 711a-c may be configured as self-expanding enclosures and may include a braided lattice of wires (e.g., nitinol wires). Unless otherwise noted, the plurality of enclosures 711a-c may include any aspect of other enclosures described herein. In some embodiments, the braided lattice of nitinol wires may include two sizes of wires braided into the lattice. For example, the braided lattice of nitinol wires formed the plurality of enclosures 711a-c may include a first portion of nitinol wires having a first diameter and a second portion of nitinol wires having a second diameter larger than the first diameter of the first portion of nitinol wires. The plurality of enclosures 711a-c including the braided lattice having the first portion of nitinol wires and the second portion of nitinol wires are sized and dimensioned and the such that when deployed within the blood vessel 102, the plurality of enclosures form an at least partially nested configuration (shown in FIG. 7D). More specifically, the second portion of wires (having the second diameter larger than the first diameter of the first portion of nitinol wires) may include a potential energy (e.g., stored energy) sufficient shorten the embolic structure when deployed within the blood vessel to form the at least partially nested configuration. This configuration results in a dense and short embolic structure relative to conventional embolic structures. Moreover, the embolic structure 710 includes a stiffness sufficient to resist migration after deployment in the blood vessel and dense enough to promote a thrombotic response that results in occlusion of the blood vessel.

In some embodiments, the first diameter of the first portion of nitinol wires having the smaller diameter may be about 0.0008 inches to about 0.00125 inches, and the second diameter of the second portion of nitinol wires having the larger diameter may be about 0.0015 inches to about 0.0025 inches. In some embodiments, the first diameter of the first portion of nitinol wires having the smaller diameter may be about 0.0008 inches to about 0.001 inches, about 0.001 inches to about 0.00125 inches, about 0.0008 inches, about 0.001 inches, or about 0.00125 inches. In some embodiments, the second diameter of the second portion of nitinol wires having the larger diameter may be about 0.0015 inches to about 0.002 inches, about 0.002 inches to about 0.0025 inches, about 0.0015 inches, about 0.002 inches, or about 0.0025 inches.

In the unconstrained, manufactured state illustrated in FIGS. 7A-7C, each of the plurality of enclosures 711a-c includes a first portion that extends generally perpendicular from the axis of the embolic structure 710 and a second portion that angles (e.g., generally perpendicular) from the first portion of each of the plurality of enclosures 711a-c. This configuration forms a nest shape such that a second enclosure 711b at least partially nests within a first enclosure 711a, and a third enclosure 711c at least partially nests within the second enclosure 711b. Each of the plurality of enclosures 711a-c may be separated or spaced from one another by a necked down portion of the braided lattice. As illustrated in FIGS. 7A-C, in the manufactured, unconstrained state, the second enclosure 711b may protrude or extend beyond a rim of the first enclosure 711a when at least partially nested therein. The third enclosure 711c may similarly protrude or extend beyond a rim of the second enclosure 711b when at least partially nested therein in the manufactured, unconstrained state. The embolic structure 710 illustrated in FIGS. 7A-7C includes three enclosures 711a-c. In other embodiments, the embolic structure 710 may include only two enclosures configured to at least partially nest within one another when deployed. In still other embodiments, the embolic structure may include four or more enclosures configured to at least partially nest within one another when deployed.

Turning now specifically to FIG. 7D, when the embolic structure 710 is deployed in the blood vessel 102, the plurality of enclosures 711a-c at least partially nest within one another in the blood vessel, such that the embolic structure 710 includes a shorter length while still having a desired density of braids in the plurality of enclosures 711a-c to provide the desired reduction of blood flow in the blood vessel 102. When deployed in the blood vessel 102, each of the plurality of enclosures 711a-c may take a generally conical or frustoconical shape having a v-shaped side profile illustrated in FIG. 7D. Accordingly, each of the plurality of enclosures 711a-c may include a narrow distal end and a wide proximal end, with each of the plurality of enclosures 711a-c defining an interior region extending between the wide proximal end and the narrow distal end. The narrow distal end of the second enclosure 711b may be at least partially nested within the interior region of the first enclosure 711a, while the wide proximal end of the second enclosure 711b may be positioned outside the interior region of the first enclosures 711a (e.g., the wide proximal end of the second enclosure 711b is not positioned between the narrow distal end and the wide proximal end of the first enclosure 711a). Similarly, the narrow distal end of the third enclosure 711c may be at least partially nested within the interior region of the second enclosure 711b, while the wide proximal end of the third enclosure 711c may be positioned outside the interior region of the second enclosures 711b (e.g., the wide proximal end of the third enclosure 711c is not positioned between the narrow distal end and the wide proximal end of the second enclosure 711b).

The embolization device including the embolic structure 710 may include any aspect of other embolization devices described herein such as a clamp 113 at a distal end, a threaded coupling 116 at the proximal end that can be threadingly coupled to a threaded end of the placement wire 130. In some embodiments, a radiopaque marker may be disposed on or incorporated with at least one of the clamp 113 and/or the threaded coupling 116. When deployed the embolic structure 710 can be rotationally held in place relative to the placement wire 130 when the embolic structure 110 engages with the vessel wall and the placement wire 130 can be rotated to release the placement wire 130 from the embolic structure 110.

Also contemplated herein is a method of restricting blood flow within a blood vessel using any of the embolic structures configured to at least partially nest within one another described herein. For example, a method of restricting blood flow within a blood vessel 102 may include positioning an embolization device into the blood vessel 102 adjacent to a treatment site. The method also may include deploying the embolization device from a delivery catheter into the blood vessel 102 at the treatment site to dispose a plurality of enclosures 711a-c of the embolization device in the blood vessel. The plurality of enclosures may include a braided lattice of nitinol wires including a first portion of nitinol wires having a first diameter and a second portion of nitinol wires having a second diameter larger than the first diameter of the first portion of nitinol wires. The method also may include at least partially nesting the second enclosure 711b in the first enclosure 711a with the first enclosure 711a and the second enclosure 711b contacting a wall of the blood vessel 102. The method also may include restricting blood flow through the embolic structure 710. In the method, the plurality of enclosures 711a-c include a stiffness to resist migration in the blood vessel 102 and a density sufficient to promote a thrombotic response resulting in occlusion of the blood vessel 102.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

References to approximations are made throughout this specification, such as by use of the term “about.” For each such reference, it is to be understood that, in some embodiments, the value, feature, or characteristic may be specified without approximation. For example, where a qualifier such as “about” is used, this term includes within its scope the qualified words in the absence of its qualifiers. For example, where the term “about” is recited with respect to a feature, it is understood that in further embodiments, the feature can have a precise configuration.

The phrase “coupled to” refer to any form of interaction between two or more entities, including mechanical, electrical, magnetic, electromagnetic, fluid, and thermal interaction. Two components may be coupled to each other even though they are not in direct contact with each other. For example, two components may be coupled to each other through an intermediate component.

The directional terms “distal” and “proximal” are given their ordinary meaning in the art. That is, the distal end of a medical device means the end of the device furthest from the practitioner during use. The proximal end refers to the opposite end, or the end nearest the practitioner during use.

The terms “a” and “an” can be described as one, but not limited to one. For example, although the disclosure may recite a housing having “a stopper,” the disclosure also contemplates that the housing can have two or more stoppers.

Unless otherwise stated, all ranges include both endpoints and all numbers between the endpoints.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Similarly, in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. An embolization device, comprising:
an embolic structure comprising a plurality of enclosures comprising a braided lattice of nitinol wires, at least one enclosure of the plurality of enclosures including an inner layer of the braided lattice of nitinol wires, an outer layer of the braided lattice of nitinol wires, and a cup-shaped member disposed in the at least one enclosure between the inner layer and the outer layer of the braided lattice of nitinol wires.

2. The embolization device of claim 1, wherein the cup-shaped member includes a material including at least one of expanded polytetrafluoroethylene, fabric, polyester, nylon, or polyether block amide.

3. The embolization device of claim 1, wherein the at least one enclosure includes a first end and a second end, and the cup-shaped member extends from the first end at least halfway to the second end.

4. The embolization device of claim 1, wherein the at least one enclosure is generally spherical and the cup-shaped member is generally semispherical.

5. The embolization device of claim 1, wherein: the plurality of enclosures includes a first enclosure and a second enclosure, the first enclosure including the inner layer of the braided lattice of nitinol wires, the outer layer of the braided lattice of nitinol wires, and the cup-shaped member disposed in the first enclosure between the inner layer and the outer layer of the braided lattice of nitinol wires.

6. The embolization device of claim 5, wherein the cup-shaped member disposed in the first enclosure includes an open end oriented towards the second enclosure.

7. The embolization device of claim 6, wherein the plurality of enclosures includes a third enclosure, the second enclosure being disposed between the first enclosure and the third enclosure, and the cup-shaped member being absent from the second enclosure and the third enclosure.

8. The embolization device of claim 6, wherein the plurality of enclosures includes a third enclosure, the first enclosure being disposed between the third enclosure and the second enclosure, and the cup-shaped member being absent from the second enclosure and the third enclosure.

9. The embolization device of claim 6, wherein:
the plurality of enclosures includes a third enclosure including an inner layer of the braided lattice of nitinol wires, an outer layer of the braided lattice of nitinol wires, and a cup-shaped member disposed in the third enclosure between the inner layer and the outer layer of the braided lattice of nitinol wires of the third enclosure;
the first enclosure is disposed between the third enclosure and the second enclosure; and
the cup-shaped member of the third enclosure includes an open end oriented towards the first enclosure.

10. The embolization device of claim 6, wherein the second enclosure includes an inner layer of the braided lattice of nitinol wires, an outer layer of the braided lattice of nitinol wires, and a cup-shaped member disposed in the second enclosure between the inner layer and the outer layer of the braided lattice of nitinol wires of the second enclosure with an open end of the cup-shaped member of the second enclosure orientated away from the first enclosure.

11. The embolization device of claim 5, wherein the cup-shaped member disposed in the first enclosure includes an open end oriented away from the second enclosure, the cup-shaped member being absent from the second enclosure.

12. A method of restricting blood flow within a blood vessel, the method comprising:
deploying an embolization device from a delivery catheter into the blood vessel at a treatment site;
disposing a plurality of enclosures of an embolic structure of the embolization device within the blood vessel, wherein the plurality of enclosures comprise a braided lattice of nitinol wires that contact a wall of the blood vessel, at least one enclosure of the plurality of enclosures including an inner layer of the braided lattice of nitinol wires, an outer layer of the braided lattice of nitinol wires, and a cup-shaped member disposed in the at least one enclosure between the inner layer and the outer layer of the braided lattice of nitinol wires, wherein the cup-shaped member includes an open end oriented away from a direction of blood flow in the blood vessel; and
restricting blood flow through the embolic structure.

13. The method of claim 12, wherein the cup-shaped member includes a material including at least one of expanded polytetrafluoroethylene, fabric, polyester, nylon, or polyether block amide.

14. The method of claim 12, wherein the at least one enclosure includes a first end and a second end, and the cup-shaped member extends from the first end at least halfway to the second end.

15. The method of claim 12, wherein the plurality of enclosures includes a first enclosure and a second enclosure, the first enclosure including the inner layer of the braided lattice of nitinol wires, the outer layer of the braided lattice of nitinol wires, and the cup-shaped member disposed in the first enclosure between the inner layer and the outer layer of the braided lattice of nitinol wires.

16. The method of claim 15, wherein the open end of the cup-shaped member disposed in the first enclosure includes is oriented towards the second enclosure.

17. The method of claim 16, wherein the plurality of enclosures includes a third enclosure, the second enclosure being disposed between the first enclosure and the third enclosure, and the cup-shaped member being absent from the second enclosure and the third enclosure.

18. The method of claim 16, wherein the plurality of enclosures includes a third enclosure, the first enclosure being disposed between the third enclosure and the second enclosure, and the cup-shaped member being absent from the second enclosure and the third enclosure.

19. The method of claim 16, wherein:
the plurality of enclosures includes a third enclosure including an inner layer of the braided lattice of nitinol wires, an outer layer of the braided lattice of nitinol wires, and a cup-shaped member disposed in the third enclosure between the inner layer and the outer layer of the braided lattice of nitinol wires of the third enclosure;
the first enclosure is disposed between the third enclosure and the second enclosure; and
the cup-shaped member of the third enclosure includes an open end oriented towards the first enclosure.

20. The method of claim 16, wherein the cup-shaped member disposed in the first enclosure includes an open end oriented away from the second enclosure, the cup-shaped member being absent from the second enclosure.
